(19) United States
(12) Patent Application Publication
(10) Pub. No.: US 2017/0182337 A1
(43) Pub. Date: Jun. 29, 2017

(54) GUIDE FOR RADIOACTIVE PARTICLE IMPLANTATION IN ONCOTHERAPY AND METHOD THEREOF

(71) Applicant: Shanghai Xinjian Medical Co., LTD., Shanghai City (CN)

(72) Inventor: FEI LIU, Shanghai City (CN)

(21) Appl. No.: 15/393,177

(22) Filed: Dec. 28, 2016

(30) Foreign Application Priority Data
Dec. 28, 2015 (CN) 201510995138.4

(51) Int. Cl. A61N 5/10

(52) U.S. Cl. CPC A61N 5/1007; A61N 5/1039; A61N 5/1027; A61N 2005/1012

(57) ABSTRACT

A method of making a guide for radioactive particle implantation in oncotherapy is disclosed. The guide making by the method has a simple structure and can guide the surgeon to carry out the radioactive particle implantation for improving the accuracy of the positions of the implanted radioactive particles and saving the time of the surgery and reducing the risk of inflection during operation.

### BACKGROUND OF THE INVENTION

### 1. Field of the Invention

The present invention relates to medical devices for oncotherapy, and more particularly to a guide for radioactive particle implantation in oncotherapy. The present invention also relates to a method of making the guide for radioactive particle implantation in oncotherapy.

### 2. Description of the Prior Art

Percutaneous puncture is a surgical method often used in tumor interventional therapy to achieve the purpose of killing the tumor cells by injecting drugs into the tumor or implanting the radioactive particles or magnetic heat seeds into the tumor through the needles. Accurately positioning during puncture plays a decisive role in the success rate of puncture. The operations of finding puncturing points by the experiences of the surgeons and punching directly the needles into the lesion in the past are inaccurate and easily affect the treatment effect caused by the deviations of the implanting positions and the uneven distribution of the radioactive particles or magnetic heat seeds due to the reasons such as the deviation of the positions and directions of the needles going into the lesion and the mistakes generated by repeated puncture during the implantation.

With the continuous development of the medical imaging technology, a method of percutaneous puncture under the guidance of the CT scanning equipment, or the ultrasound scanning equipment or the magnetic resonance image scanning equipment is already appeared. The method includes following steps: firstly determining the puncturing points and the puncturing depths by the imaging equipment such as a CT scanner; secondly carrying out the tumor puncture based on the puncturing points and the puncturing depths through the experience of the surgeon; and lastly implanting the radioactive particles or magnetic heat seeds into the tumor. The method can greatly improve the accuracy and safety of the puncture, however the defects of inaccurate positioning which is caused by the deviations of the position and direction of the surgeon's hand and the depths accurately determined through repeatedly puncturing are existing because the guidance of the CT scanner is not a real-time guide.

Another method for percutaneous puncture through conventional template interpolation under the guidance of the CT scanner or ultrasound scanning equipment includes following steps: firstly determining the punching points and the punching depths through the guidance of the CT scanner; then placing the conventional template onto the surface of the implanted portion according to the punching points and the punching depths; lastly carrying out the implantation of the radioactive particles or magnetic heat seeds into the implanted portion by the needle or implanting gun held by a surgeon and passing through the equal-spaced holes of the conventional template. The accuracy of the particle implantation is improved, however, the deviations of the positions or the angle between the conventional template and the patient easily appear in the complicated anatomical structure and results in the needle inserting paths and the inserting positions during the implantation being inconsistent with that in plan, so as to reduce the accuracy of treatment and the radiation dose of the tumor, and increase the radiation dose of the normal tissue and the complications, and affect the treatment.

Accordingly, there is a need in the art for improved guide for radioactive particle implantation in oncotherapy and method thereof.

### SUMMARY OF THE INVENTION

Accordingly, an object of the present invention is to provide a guide for a surgeon to carry out radioactive particle implantation in oncotherapy, so as to improve the accuracy of the implanting positions of the radioactive particles. The object of the present invention is also to provide a method of making the guide for radioactive particle implantation.

In order to achieve the object set forth, a method of making a guide for radioactive particle implantation in oncotherapy comprises following steps: scanning a predetermined portion of the patient through a scanner to obtain a medical image of the predetermined portion; obtaining an image of an interest region from the medical image, the interest region including a lesion portion and tissue portions associated with the lesion portion; reconstructing the image data of the interest region to obtain a 3D model of the interest region; determining virtual paths which allow the needle going to the lesion portion based on the 3D model of the interest region; determining virtual positions, virtual directions and virtual depths based on the virtual paths; obtaining a 3D model of a guide for radioactive particle implantation based on the 3D model of the interest region, the virtual paths, the virtual positions and the virtual directions; and obtaining the guide for radioactive particle implantation in oncotherapy through manufacturing the 3D model by the rapid prototyping technology.

In order to achieve the object set forth, a guide making by the method described above comprises a base having a plurality of through holes extending along the thickness direction thereof and a plurality of guiding portions extending from a peripheral portion of the through hole.

Other objects, advantages and novel features of the present invention will become more apparent from the following detailed description when taken in conjunction with the accompanying drawings, in which:

### BRIEF DESCRIPTION OF THE DRAWINGS

FIG. 1 is a flowchart depicting a method of making a guide for radioactive particle implantation in oncothreapy in accordance with the preferred embodiment of the present invention;

FIG. 2 is a flowchart of step 13 for making a 3D model of the interesting portion shown in FIG. 1;

FIG. 3 is a flowchart of step 14 for obtaining a virtual path along which the surgical instruments with radioactive particles going to a predetermined portion shown in FIG. 1;

FIG. 4 is a flowchart of step 16 for obtaining a 3D model of the guide for radioactive particle implantation in oncotherapy shown in FIG. 1;

FIGS. 5a-5c illustrate CT images obtained by scanning the hip of the patient in accordance with the preferred embodiment of the present invention;

FIG. 6 illustrates a 3D model of the focus position reconstructed from the CT images shown in FIG. 5a-5c according to the method described in FIG. 1;

FIGS. 7a-7b illustrate virtual paths along which the surgical instruments with radioactive particles going to the focus position shown in FIG. 6 according to the method of the present invention;

FIG. 8 is an assemble, perspective view showing a 3D model of the guide for radioactive particle implantation assembled on the 3D model of the interesting portion, and the 3D model of the guide for radioactive particle implantation obtained according to the method shown in FIG. 4; and

FIG. 9 is a perspective view of the guide for radioactive particle implantation according to the method of the present invention.

### DESCRIPTION OF PREFERRED EMBODIMENTS OF THE INVENTION

Reference is now made to the drawings to describe the invention in detail.

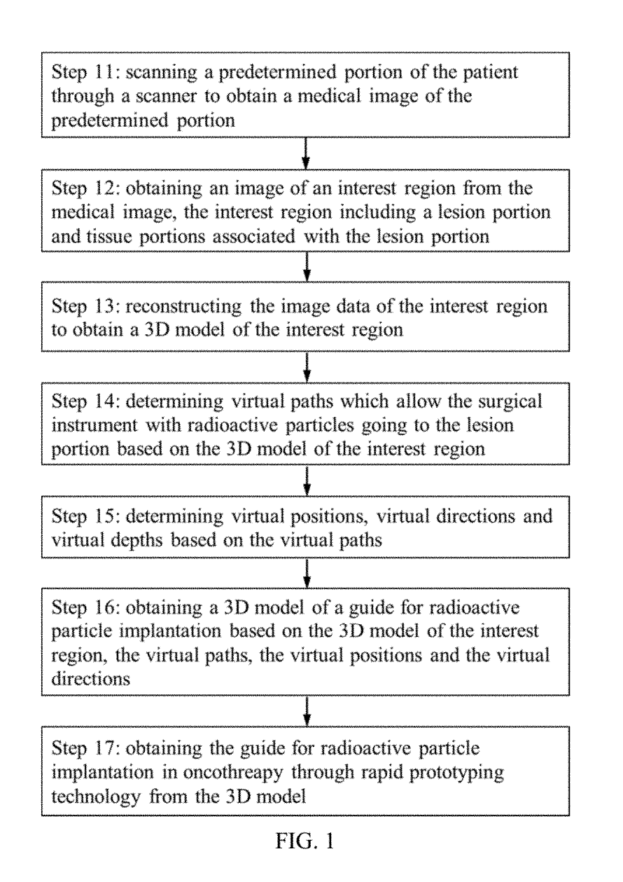

FIG. 1 illustrates a method of making a guide for radioactive particle implantation in oncothreapy in accordance with the preferred invention. The method comprises following steps:

Step 11: scanning a predetermined portion of the patient through a scanner to obtain a medical image of the predetermined portion;

Step 12: obtaining an image of an interest region from the medical image, the interest region including a lesion portion and tissue portions associated with the lesion portion;

Step 13: reconstructing the image data of the interest region to obtain a 3D model of the interest region;

Step 14: determining virtual paths which allow the needle going to the lesion portion based on the 3D model of the interest region;

Step 15: determining virtual positions, virtual directions and virtual depths based on the virtual paths;

Step 16: obtaining a 3D model of a guide for radioactive particle implantation based on the 3D model of the interest region, the virtual paths, the virtual positions and the virtual directions; and

Step 17: obtaining the guide for radioactive particle implantation in oncothreapy through rapid prototyping technology from the 3D model.

Specifically, in step 11, the scanner is a computed tomography scanning equipment or a magnetic resonance imaging equipment or a PET-CT scanning equipment. Referring to FIGS. 5a-5c, the predetermined portion is the hip of the patient, and the medical image is a CT image obtained through the CT scanner scanning the hip of the patient. FIG. 5a illustrates a cross-sectional image of the hip obtained by the CT scanner, FIG. 5b illustrates a coronal image of the hip obtained by the CT scanner, and FIG. 5c illustrates a sagittal image of the hip obtained by the CT scanner.

In step 13, the image data of the interest region is obtained by segmenting the image data of the predetermined portion according to tissue portions, and the 3D model of the interest region is obtained through respectively reconstructing the image data of the interest region which are segmented from the image of the predetermined portion according to tissue portions. The 3D model of the interest region including the 3D model of the lesion portion and the 3D model of the tissue portions associated with the lesion portion. In step 14, the virtual paths are determined by the shape and size of the 3D model of the lesion portion, and the total dose of the implanted radioactive particles which is determined by the shape and size of the 3D model of the lesion portion.

In step 15, the virtual directions and the virtual positions are obtained according to the virtual paths which allow the needle going to the lesion portion and the outer surface of the 3D model of the interest region. The virtual depths are obtained according to the virtual paths and the distribution locations of the radioactive particles. The distribution locations of the radioactive particles are determined through uniformly distributing the total implanted radioactive particles in the 3D model of the lesion portion.

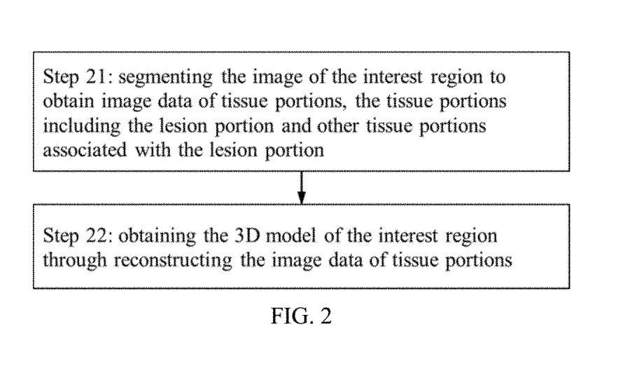

FIG. 2 illustrates a method of obtaining the 3D model of the interest region in step 13. The method includes following steps:

Step 21: segmenting the image of the interest region to obtain image data of tissue portions, the tissue portions including the lesion portion and other tissue portions associated with the lesion portion; and

Step 22: obtaining the 3D model of the interest region through reconstructing the image data of tissue portions.

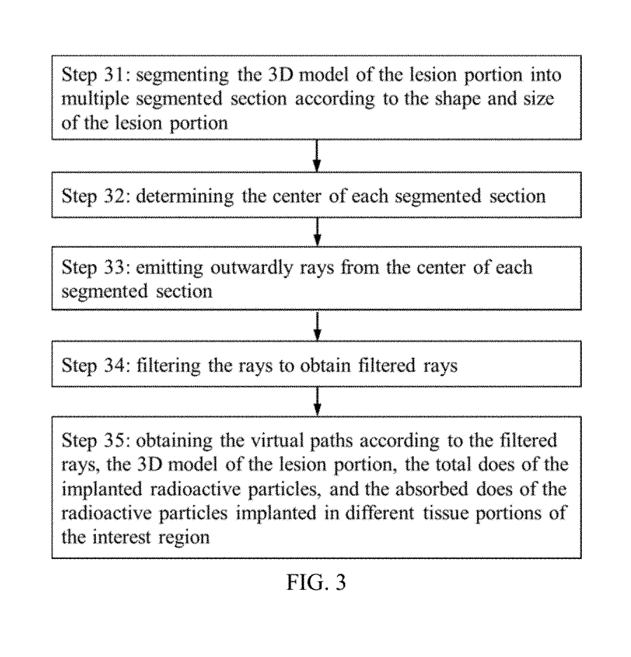

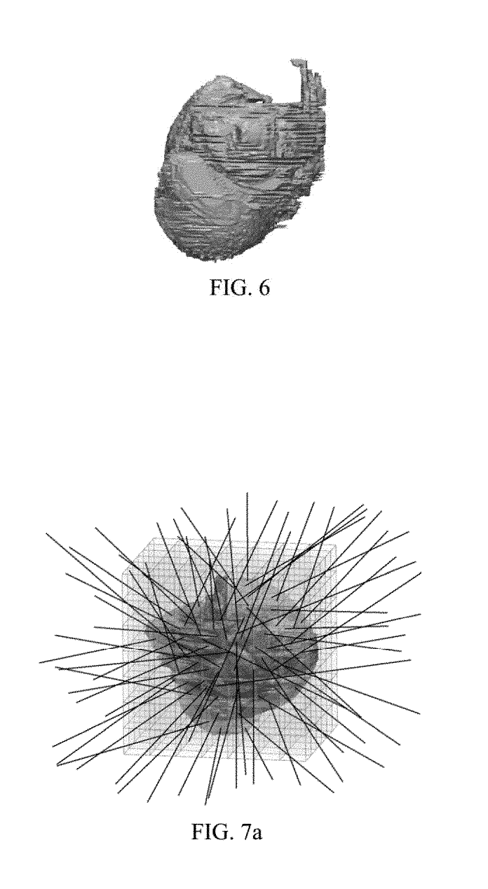

FIG. 3 illustrates a method of determining the virtual paths in step 14. Referring to FIGS. 6, 7a and 7b, and 8, the method for determining the virtual paths according to the shape and size of the 3D model of the lesion portion and the total does of the implanted radioactive particles includes following steps:

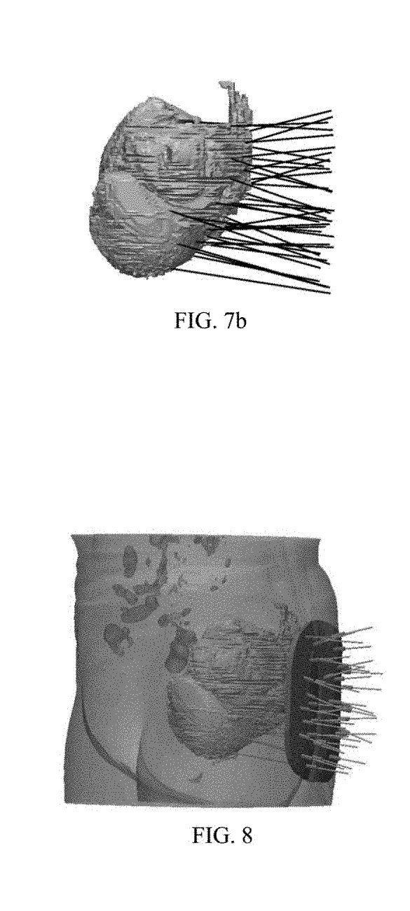

Step 31: segmenting the 3D model of the lesion portion into multiple segmented section according to the shape and size of the lesion portion, referring to FIG. 7a;

Step 32: determining the center of each segmented section;

Step 33: emitting outwardly rays from the center of each segmented section;

Step 34: filtering the rays to obtain filtered rays; and

Step 35: obtaining the virtual paths according to the filtered rays, the 3D model of the lesion portion, the total does of the implanted radioactive particles, and the absorbed does of the radioactive particles implanted in different tissue portions of the interest region.

Specifically, in step 34, the rays are filtered base on whether the rays fall within the range of the operating orientation in surgery and pass through the 3D model of the important tissue portion in the interest region. The operating orientation is determined by the patient position. The ray passing through the 3D model of the important tissue portion is deleted. The important tissue portion is an important organ, or a blood vessel or a nerve. The ray falling a portion out of the range of the operating orientation is deleted. The filtered rays are the virtual paths. FIG. 7b illustrates the virtual paths allow the needle going to the sacral tumor according to the method of the present invention.

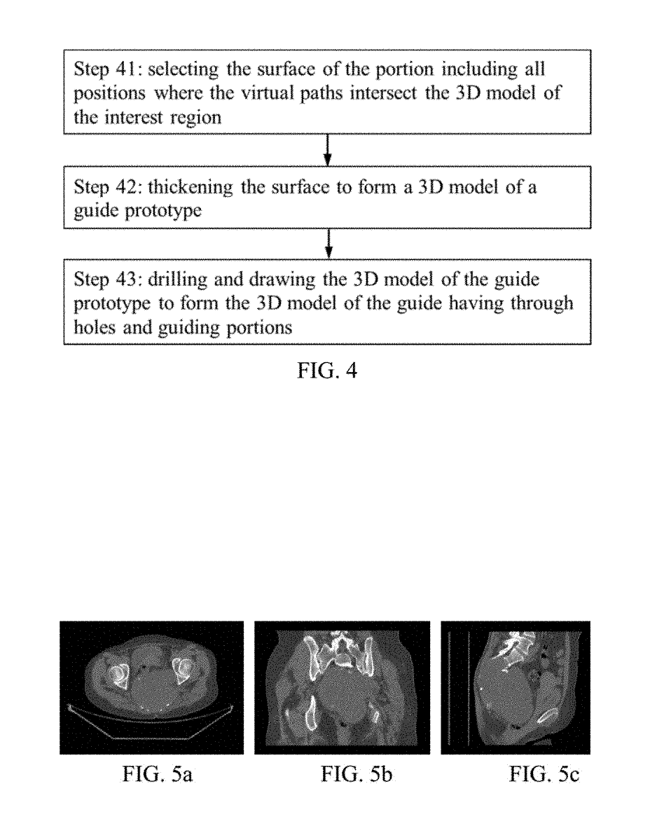

FIG. 4 illustrates a method of obtaining a 3D model of the guide for radioactive particle implantation in oncotherapy in step 16. The method includes following steps:

Step 41: selecting the surface of the portion including all positions where the virtual paths intersect the 3D model of the interest region;

Step 42: thickening the surface to form a 3D model of a guide prototype; and

Step 43: drilling and drawing the 3D model of the guide prototype to form the 3D model of the guide having through holes and guiding portions.

Specifically, in step 43, the through holes of the 3D model of the guide are formed through drilling the portions of the 3D model of the guide prototype corresponding to the virtual positions. The extending direction of each through hole is consistent with the corresponding virtual direction. The size of each through hole is determined by the size of the corresponding needle for implanting radioactive particles during the radioactive particle implantation. The distance between adjacent through holes is set based on the distance between corresponding virtual paths along which the needles go to the lesion portion. The guiding portions of the 3D model of the guide are formed through drawing the portions around the through holes. The guiding portions respectively extend along the corresponding virtual directions.

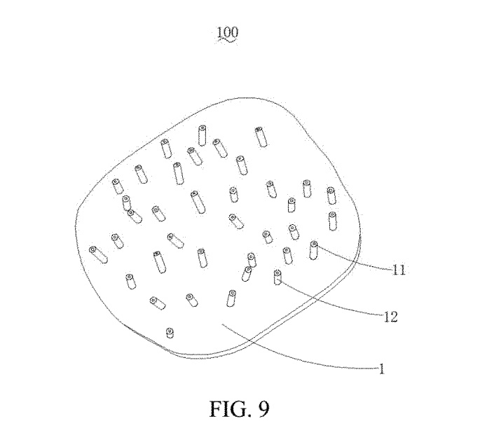

FIG. 9 illustrates a guide 100 for radioactive particle implantation in surgical treatment of sacrum tumor according to the method of the present invention. The guide 100 integrated through rapid prototyping technology includes a base 1 disposed to a surface of the corresponding portion of the patient. The base 1 defines a plurality of through holes 11 extending along the thickness direction thereof. The distance between two adjacent through holes 11 is determined by the distance between the corresponding virtual paths. The extending direction of each though hole 11 is consistent with the extending direction of the corresponding virtual path. The base 1 has a plurality of guiding portions 12 extending from a peripheral portion of the through hole 11. Each guiding portion 12 configured with a hollow cylinder shape extends along the extending direction of the through hole 11. The sacrum tumor in the preferred embodiment of the present invention has a size of 130 millimeters by 107 millimeters by 85 millimeters. The size of the sacrum tumor is measured by a CT scanner.

The guide made according to the method of the present invention can guide radioactive particles accurately into the lesion portions of the patient through the needles, so as to ensue radioactive particles evenly distributed in the lesion portion and avoid the therapeutic effect which is resulted in uneven distribution of the radioactive particles caused by the deviations of the directions and positions of the needles during the traditional oncology treatment surgery. The guide made by the method of the present invention also can avoid the problem of repeatedly punching the needles resulted by the different size, different shape of different tumor in traditional surgery and improve the precision rate of the radioactive particle implanting positions and the radioactive particle distribution, so as to achieve the purpose of personalized medicine. In addition, the guiding portions of the guide integrated through the rapid prototyping technology are determined by the directions of the needle, so as to simplify the operation process during the radioactive particle implantation for saving the time of the surgery and reducing the risk of inflection during operation. Furthermore, the guide for radioactive particle implantation in oncotherapy according to the method of the present invention is configured with a simple construction and easily manufactured.

Furthermore, although the present invention has been described with reference to particular embodiments, it is not to be construed as being limited thereto. Various alterations and modifications can be made to the embodiments without in any way departing from the scope or spirit of the present invention as defined in the appended claims.

What is claimed is:

1. A method of making a guide for radioactive particle implantation in oncotherapy comprising:
a) scanning a predetermined portion of the patient through a scanner to obtain a medical image of the predetermined portion;
b) obtaining an image of an interest region from the medical image, the interest region including a lesion portion and tissue portions associated with the lesion portion;
c) reconstructing the image data of the interest region to obtain a 3-dimensional (3D) model of the interest region;
d) determining virtual paths which allow the needle going to the lesion portion based on the 3D model of the interest region;
e) determining virtual positions, virtual directions and virtual depths based on the virtual paths;
f) obtaining a 3D model of a guide for radioactive particle implantation based on the 3D model of the interest region, the virtual paths, the virtual positions and the virtual directions; and
g) obtaining the guide for radioactive particle implantation in oncotherapy through manufacturing the 3D model by the rapid prototyping technology.

2. The method of claim 1, wherein the image data of the interest region is obtained by segmenting the image data of the predetermined portion according to tissue portions, and wherein the 3D model of the interest region is obtained through respectively reconstructing the image data of the interest region which are segmented from the image of the predetermined portion according to tissue portions.

3. The method of claim 1, wherein a method of obtaining the 3D model of the interest region including:
a) segmenting the image of the interest region to obtain image data of tissue portions, the tissue portions including the lesion portion and other tissue portions associated with the lesion portion; and
b) obtaining the 3D model of the interest region through reconstructing the image data of tissue portions.

4. The method of claim 3, wherein the 3D model of the interest region includes the 3D model of the lesion portion and the 3D model of the tissue portions associated with the lesion portion.

5. The method of claim 4, wherein a method of determining virtual paths based on the 3D model of the interest region including:
a) determining the total dose of the implanted radioactive particles based on the shape and size of the 3D model of the lesion portion; and
b) determining virtual paths according to the shape and size of the 3D model of the lesion portion and the total dose of the implanted radioactive particles.

6. The method of claim 5, wherein a method of determining virtual paths according to the shape and size of the 3D model of the lesion portion and the total dose of the implanted radioactive particles including:
a) segmenting the 3D model of the lesion portion into multiple segmented section according to the shape and size of the lesion portion;
b) determining the center of each segmented section;
c) emitting outwardly rays from the center of each segmented section;
d) filtering the rays to obtain filtered rays;
e) obtaining the virtual paths according to the filtered rays, the 3D model of the lesion portion, the total does of the implanted radioactive particles, and the absorbed does of the radioactive particles implanted in different tissue portions of the interest region.

7. The method of claim 6, wherein the virtual directions and the virtual positions are obtained according to the virtual paths which allow the needle going to the lesion portion and the outer surface of the 3D model of the interest region.

8. The method of claim 6, wherein the virtual depths are obtained according to the virtual paths and the distribution locations of the radioactive particles, and wherein the distribution locations of the radioactive particles are determined through uniformly distributing the total implanted radioactive particles in the 3D model of the lesion portion.

9. The method of claim 6, wherein the rays are filtered base on whether the rays fall within the range of the operating orientation in surgery and whether the rays pass through the 3D model of the important tissue portion in the interest region.

10. The method of claim 9, wherein the operating orientation is determined by the patient position, and wherein the ray passing through the 3D model of the important tissue portion is deleted, and wherein the important tissue portion is an important organ, or a blood vessel or a nerve.

11. The method of claim 10, wherein the ray falling a portion out of the range of the operating orientation is deleted.

12. The method of claim 1, a method of obtaining a 3D model of a guide for radioactive particle implantation based on the 3D model of the interest region, the virtual paths, the virtual positions and the virtual directions including:
a) selecting the surface of the portion including all positions where the virtual paths intersect the 3D model of the interest region;
b) thickening the surface to form a 3D model of a guide prototype; and
c) drilling and drawing the 3D model of the guide prototype to form the 3D model of the guide having through holes and guiding portions.

13. The method of claim 12, wherein the through holes of the 3D model of the guide are formed through drilling the portions of the 3D model of the guide prototype, and wherein the portions of the 3D model of the guide prototype respectively correspond to the virtual positions, and wherein the extending direction of each through hole is consistent with the corresponding virtual direction.

14. The method of claim 13, wherein the size of each through hole is determined by the size of the corresponding needle for implanting radioactive particles during the radioactive particle implantation, and wherein the distance between adjacent through holes is set based on the distance between corresponding virtual paths along which the needles go to the lesion portion.

15. The method of claim 12, wherein the guiding portions of the 3D model of the guide are formed through drawing the portions around the through holes, and wherein the guiding portions respectively extend along the corresponding virtual directions.

16. A guide making by the method of claim 10 for radioactive particle implantation in oncotherapy comprising a base having a plurality of through holes extending along the thickness direction thereof and a plurality of guiding portions extending from a peripheral portion of the through hole.

17. The guide of claim 16, wherein the guide is integrated by rapid prototyping technology, and wherein the extending direction of the through hole is consistent with that of the corresponding guiding portion.

18. A guide making by the method of claim 12 for radioactive particle implantation in oncotherapy comprising a base having a plurality of through holes extending along the thickness direction thereof and a plurality of guiding portions extending from a peripheral portion of the through hole.

19. The guide of claim 18, wherein the guide is integrated by rapid prototyping technology, and wherein the extending direction of the through hole is consistent with that of the corresponding guiding portion.
